(19) United States
(12) Patent Application Publication
(10) Pub. No.: US 2016/0045127 A1
(43) Pub. Date: Feb. 18, 2016

(54) AUTOMATED DETECTOR AND CLASSIFIER OF HIGH FREQUENCY OSCILLATIONS AND INDICATOR SEIZURE ONSET

(71) Applicant: THE REGENTS OF THE UNIVERSITY OF MICHIGAN, Ann Arbor, MI (US)

(72) Inventors: William C. Stacey, Saline, MI (US); Stephen Gliske, Ann Arbor, MI (US)

(21) Appl. No.: 14/825,826

(22) Filed: Aug. 13, 2015

Related U.S. Application Data
(60) Provisional application No. 62/037,934, filed on Aug. 15, 2014.

(51) Int. Cl. A61B 5/048; A61N 1/36; A61B 5/00; A61B 5/04; A61B 5/0478

(52) U.S. Cl. CPC A61B 5/048; A61B 5/04001; A61B 5/0478; A61B 5/04012; A61B 5/4094; A61N 1/36064; A61N 1/36135; A61B 5/742

(57) ABSTRACT

High frequency oscillations (HFOs) are automatically detected in electroencephalogram (EEG) signals and analyzed to assess whether they are predictive of the onset of a neurological dysfunction in a subject or an indication of nonneurological electrical activity or noise in the EEG signal. In some examples, HFOs, serving as a biomarker for epileptic seizures, are identified and used to identify seizure networks within a patient for clinician monitoring or for controlling automated treatment systems. The analysis may be used to create enhanced EEG displays, with HFOs identified on the EEG.

### CROSS-REFERENCE TO RELATED APPLICATIONS

This application claims the priority benefit under 35 U.S.C. §119(e) of U.S. Provisional Patent Application No. 62/037,934, filed Aug. 15, 2014, the disclosure of which is incorporated herein by reference in its entirety.

### STATEMENT REGARDING FEDERALLY SPONSORED RESEARCH OR DEVELOPMENT

This invention was made with government support under NS069783 and TR000433 awarded by the National Institutes of Health. The Government has certain rights in the invention.

### FIELD OF THE DISCLOSURE

The present disclosure relates generally to techniques for analyzing electroencephalogram (EEG) signals and, more particularly, to techniques for analyzing EEG signals to identify and classify high frequency oscillations indicating seizure onset, and to techniques for automatically determining which EEG electrodes are within the seizure onset zone.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Epilepsy clinicians and researchers are actively searching for new tools to help identify seizure networks. Clinical care still relies upon reading electroencephalograms (EEGs), using techniques that were developed in the 1930s. The foundation of these techniques is visual interpretation of EEG signals by a clinician, typically viewing more than 20 channels with 10 seconds of real-time data on a single page at a time. Over the past 20 years, researchers have discovered that higher resolution, research grade EEG identifies new signals that were never seen before and have strong correlation with epilepsy. The most well-known of these new biomarkers of epilepsy are High Frequency Oscillations (HFOs).

There are a handful of suggested techniques of HFO analysis for epileptic seizure analysis. However, HFO analysis stills remain isolated; and the current techniques require advanced technology, expertise, and are not sufficiently automated. For example, standard EEG displays and analytic techniques are incapable of viewing or identifying HFOs. Physicians can attempt to use manual methods for HFO detection, but this is impractical for clinician usage. In order to translate the HFO biomarker into clinical practice, automated tools for identifying and locating HFOs in EEG signals are desired.

One of the largest impediments to HFO detection algorithms has been the unreliability and inconsistency of the data collection schemes, schemes that are hampered, in part, by the large amounts of data collected in an EEG. One HFO detector is called the “Staba” detector (Staba R J, Wilson C L, Bragin A, Fried I, Engel J, Jr. Quantitative analysis of high-frequency oscillations (80-500 Hz) recorded in human epileptic hippocampus and entorhinal cortex. Journal of neurophysiology. 2002; 88(4):1743-52.), which uses a band-pass filter and then searches for oscillations of sufficient length and difference from the background EEG signal, which is a combination of noise and normal brain activity. The Staba detector is highly sensitive, but quite prone to identifying ‘artifacts’ such as noise and patient movement as HFOs, when they are not. The Staba detector, for example, is particularly susceptible to incorrectly identifying (as HFOs) fast transients that produce false oscillations during filtering due to the Gibb's phenomenon. Benar C G, Chauviere L, Bartolomei F, Wendling F. Pitfalls of high-pass filtering for detecting epileptic oscillations: a technical note on “false” ripples. Clin Neurophysiol. 2010; 121(3):301-10. Despite its relative popularity, the Staba detector's noise issues are well-known and documented. A few researchers studying Staba detectors have found that using the detector on long-term human EEG requires a complicated, multi-step manual process if one hopes to eliminate even obvious artifacts from the collected data. That is, some techniques have attempted to improve upon Staba detectors by eliminating obvious artifacts, but these techniques require specific adjustments for each patient individually and cannot be done in a fully automated fashion. Further, in long term EEG there are frequently periods of poor data quality in which automated algorithms are unreliable. Further still, any algorithm must account for false positive detections from transient artifacts, which no conventional techniques do. The most advanced technique first uses a manually-customized process to redact high background signals, then uses a separate classification algorithm to group together all obvious artifacts into an “artifact cluster.” (Blanco J A, Stead M, Krieger A, Viventi J, Marsh W R, Lee K H, Worrell G A, Litt B. Unsupervised classification of high-frequency oscillations in human neocortical epilepsy and control patients. Journal of neurophysiology. 2010; 104(5):2900-12). Other techniques to detect HFOs have been proposed, but none have addressed the need to remove artifacts or redact periods of poor signal quality; all of them use manual review of the detected HFOs, an exhaustive process that makes them very difficult for use in standard clinical practice. The techniques were developed and tuned to a specific dataset and set of acquisition parameters, and their use remains isolated to one or two research centers where they were developed and are not part of standard clinical practice. Another impediment to translation to clinical practice is the determination of how to use the HFO data to identify the seizure onset. In particular, recent studies have shown that HFOs are very specific to the location where a particular patient's seizures initiate, known as the ‘seizure onset zone’. These retrospective studies used 10 minute, specially selected segments of manually-detected HFOs to show a correlation between electrodes with high HFO rates and the seizure onset zone (Jacobs J, Zijlmans M, Zelmann R, Chatillon C E, Hall J, Olivier A, Dubeau F, Gotman J. High-frequency electroencephalographic oscillations correlate with outcome of epilepsy surgery. Annals of neurology. 2010; 67(2):209-20. Haegelen C, Perucca P, Chatillon C E, Andrade-Valenca L, Zelmann R, Jacobs J, Collins D L, Dubeau F, Olivier A, Gotman J. High-frequency oscillations, extent of surgical resection, and surgical outcome in drug-resistant focal epilepsy. Epilepsia. 2013; 54(5):848-57). However, there exists no procedure to determine how to utilize high HFO rates prospectively, nor to display HFOs in a fashion that allows clinicians to use them in their clinical interpretation. Because HFOs can be detected in normal as well as epileptic tissue, there are several challenges in using HFOs to identify seizures: 1) there is currently no method to determine which HFOs are due to epilepsy versus those that are due to normal brain activity; 2) it is unclear how specific features of HFOs, e.g. their frequency content, size, colocalization with other EEG signal, etc., are associated with epilepsy; 3) there is always an electrode with the highest rate, but it is not necessarily due to epilepsy; 4) it is unclear how many of the ‘highest’ channels are associated with epilepsy. Thus, a method is needed to determine a) how to distinguish ‘epileptic’ from ‘normal’ HFOs, b) how to analyze and display HFOs to provide useful information to clinicians for interpretation, c) whether the HFO rate on a given channel is indicative of epilepsy and d) how many of the other channels are also indicative of epilepsy.

Thus, there remains a desire to have an HFO detection and processing technique that can provide higher accuracy and specificity to epilepsy, as well as a method to utilize and display that information in a prospective manner to predict which channels are associated with epilepsy, and which can be done in universal algorithm agnostic to the data set.

### SUMMARY OF THE INVENTION

Techniques are described for automating HFO detection and analysis, and presenting HFO data in a form that clinicians can utilize in their typical workflow, specifically identifying for the clinicians which HFOs are associated with epilepsy. HFOs, serving as a novel biomarker for epileptic seizures, can be used to identify seizure networks within a patient and direct clinicians to monitor and treat the patient before seizure onset, or as part of an automated system to treat seizures with a closed-loop antiepilepsy device. In some examples, the HFO detection may be used in identifying seizure networks in patients undergoing surgery for epilepsy, to allow physicians to more accurately target areas for treatment or removal.

In accordance with an example, a method comprises: continuously receiving, at a signal processing device, neuronal electrical activity signal data taken from a plurality of electrodes and over a sampling window of time; forming, in the signal processing device, an optimized signal from the neuronal electrical activity signal data; identifying, in the signal processing device, windows of low-quality signal data collection within the optimized signal and removing the identified windows to form a quality-assured epochs of data collection; from the optimized signal, detecting high frequency oscillations in the optimized signal and determining a rate and/or features of high frequency oscillations over the sampling window of time, wherein the rate and/or features of high frequency oscillations are predictive of the onset of a neurological dysfunction in a subject; and from the optimized signal with detected high frequency oscillations, identifying and displaying the time, location, rate and/or features of the high frequency oscillations within a clinical viewing platform such that a physician or caregiver can visualize this additional information and incorporate it into clinical decision making.

In accordance with an example, a system comprises: a processor and a memory, the memory storing instructions that when executed by the processor, cause the processor to: continuously receive neuronal electrical activity signal data taken from a plurality of electrodes and over a sampling window of time; form an optimized signal from the neuronal electrical activity signal data; identify windows of low-quality signal data collection within the optimized signal and removing the identified windows to form a quality-assured epochs of data collection; and from the optimized signal, detect high frequency oscillations in the optimized signal and determining a rate and/or features of high frequency oscillations over the sampling window of time, wherein the rate and/or features of high frequency oscillations are predictive of the onset of a neurological dysfunction in a subject; and from the optimized signal with detected high frequency oscillations, identify and display the time, location, rate and/or features of the high frequency oscillations within a clinical viewing platform such that a physician or caregiver can visualize this additional information and incorporate it into a clinical decision making.

In accordance with an example, a method of displaying electroencephalogram signal data, the method comprises: receiving the electroencephalogram signal data; determining at least one of (i) quality-assured high frequency oscillations in the electroencephalogram signal data, (ii) insufficient quality of the signal, (iii) abnormal high frequency oscillations in the electroencephalogram signal data, abnormal high frequency oscillations being due to neurological dysfunction in a subject's brain activity, (iv) normal high frequency oscillations in the electroencephalogram signal data, normal high frequency oscillations being due to normal brain activity in the subject, and (v) seizure onset; and displaying the electroencephalogram signal data with the determination of (i), (ii), (iii), (iv), and/or (v).

In accordance with another example, a system comprises a processor and a memory, the memory storing instructions that when executed by the processor, cause the processor to: receive the electroencephalogram signal data; determine at least one of (i) quality-assured high frequency oscillations in the electroencephalogram signal data, (ii) insufficient quality of the signal, (iii) abnormal high frequency oscillations in the electroencephalogram signal data, abnormal high frequency oscillations being due to neurological dysfunction in a subject's brain activity, (iv) normal high frequency oscillations in the electroencephalogram signal data, normal high frequency oscillations being due to normal brain activity in the subject, and (v) seizure onset; and displaying the electroencephalogram signal data with the determination of (i), (ii), (iii), (iv), and/or (v).

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 illustrates an example process for automatically detecting high quality HFOs and analyzing these to develop a predictor of seizure onset (e.g., in time or location), in accordance with an example.

FIGS. 2A-2N illustrate example wave forms. FIGS. 2A-2D and FIGS. 2E-2H each show an example waveform, as they are processed using two different techniques. The raw signals are conventionally displayed using the single channel instrument reference FIGS. 2A and 2E, then in HFO analysis are bandpass filtered between 80 Hz and 500 Hz to allow the Staba detector to function (FIGS. 2C and 2G). In these examples, the Staba technique did not detect a valid HFO on the left (FIG. 2C) and incorrectly detected an artifact as an HFO on the right (FIG. 2G). In contrast, the example process herein utilizes a common average reference to display the raw signals (FIGS. 2B and 2F), which after filtering and the Staba process correctly identify an HFO (FIG. 2D) and do not label the artifact (FIG. 2H). In addition, FIGS. 2E-2H show a data segment that has low data quality based upon the large number of diffuse artifacts. The process herein uses a variety of universal artifact detectors to assess periods of data quality and to identify when individual HFO detections are likely to be due to artifacts rather than brain activity. Thus, the data would be redacted regardless of the HFO detection results. FIGS. 2I and 2N show examples of other types of artifacts for which specific detectors are implemented: bumps FIG. 2I, low signal to noise ratio FIG. 2J, high noise or recovery from flatline FIG. 2K, and pops, steps, and fast transients FIGS. 2L-2N.

FIG. 3 illustrates an example process as may be implemented by enhanced HFO detection process performed by the example process of FIG. 1. In this case, the algorithm determines the HFO and artifact detections, processes them to determine data quality, and identifies HFOs that are most likely to be due to brain activity.

FIG. 4 illustrates a process for determining a seizure onset zone from an enhanced HFO detection, in accordance with an example.

FIGS. 5A and 5B illustrate an example automated method of determining how many channels have abnormally-high rates of HFOs. FIG. 5A shows a quality-assured HFO (qHFO) rate per channel for an example preliminary prediction for patient UM-02. The horizontal line represents the threshold as determined by the procedure. In this case, 5 of the channels were determined to be over threshold. FIG. 5B shows the Kernel Density Estimator of the distribution of rates from FIG. 5A. The threshold is depicted as a vertical line and separates the peak near 3.5 qHFOs/min from the main portion of the distribution.

FIGS. 6A and 6B illustrates comparison of HFO detection methods based on the label of the channel with the highest HFO rate over all quality assured inter-ictal segments per patient. The methods are: FIG. 6A, the basic Staba HFO detector, using conventional single channel reference and FIG. 6B, the qHFO detector. The channels are labeled as either exactly matching the clinical seizure onset zone, not in the seizure onset zone (a false positive), concordant with patient outcome (i.e. resected with good outcome, but not labeled by clinicians as official seizure onset zone) or indeterminate because of insufficient patient metadata.

FIGS. 7A-7I illustrates HFO, data quality, and predictions versus time using the example depicted in part in FIG. 3. FIGS. 7A-7C correspond to patient MC-01, while FIGS. 7D-7I correspond to patient UM-02. FIG. 7A and FIG. 7D plot the mean qHFO rate and percentage of valid time (‘live fraction’) per each 10-minute epoch. Quality assured interictal times, poor quality times, and near-ictal times are indicated by colored shading, with vertical red lines for clinical seizures. FIGS. 7C, 7E and 7F demonstrate the epochs during which specific channels are predicted to be in the seizure onset zone. The algorithm identifies all predictions preliminarily, then assesses whether they are consistent over a longer period, which are labeled as semifinal predictions (purple lines indicate semifinal predictions; blue lines indicate preliminary predictions that were inconsistent). FIGS. 7B and 7G-7I are diagrams of electrode placements in each patient.

FIG. 8 illustrates the number of channels predicted to be within the seizure onset zone in each patient. There is great disparity between the number of channels predicted from patient to patient, despite the fact that the same algorithm was utilized in each case.

FIGS. 9A-9F illustrates comparison of different methods to determine how many electrodes with highest HFO rates are within the seizure onset zone for the preliminary predictions. It compares the Kernel Density Estimator (KDE) with choosing a fixed number in each patient (max−n for n=1, 2, and 3). FIGS. 9A-9D present the results for final predictions for n=1 (FIG. 9A), n=2 (FIG. 9B), n=3 (FIG. 9C) and the KDE method (FIG. 9D). FIG. 9E provides the legend for the categorization. FIG. 9F presents a histogram of the number of channels in each semi-final prediction over all semi-final predictions and patients. These results show that choosing the number of channels based upon a forced “max−n” channels is not as accurate across patients as the KDE.

FIGS. 10A-10F illustrate comparisons of examples of the present techniques against conventional techniques. FIGS. 10A and 10C show comparisons for two nominal Staba HFO techniques, highest channel and Tukey's fence, respectively. FIGS. 10B, 10D, and 10E show comparisons for qHFO techniques, highest channel, Tukey's fence, and the present techniques, respectively. FIG. 10F illustrates a comparison table across the techniques for different patients.

FIG. 11 illustrates a system for automatically detecting high quality HFOs and analyzing these to develop a predictor of seizure onset zone, in accordance with an example.

FIGS. 12A and 12B illustrate a plot of electroencephalogram (EEG) data that has been modified by the overlay of identified HFOs data, in accordance with an example.

### DETAILED DESCRIPTION

Provided are techniques for analyzing EEG signals to identify and classify high frequency oscillations indicating seizure onset. The techniques, which may be implemented in software and/or hardware and which may be fully or partially automated, offer a number of advantages including an ability to be used on top of existing HFO detection schemes (such as the Staba detector), an ability to distinguish HFOs arising from normal neural activity from those associated with seizure onset, an ability to display the HFO data to clinicians within their normal workflow, and an ability to correspondingly predict seizure onset and epileptic regions based on the rates of this more accurate class of HFOs.

The techniques involve a number of general procedures. One procedure is the enhancement and automation of HFO detection. In some examples, novel HFO detector and processing techniques are used to automatically improve the accuracy of the estimated rate of HFO detections using existing techniques. The enhancement involves, for example, identifying and removing EEG signals during times (possibly varying per channel) that are not deemed to have sufficient quality for analysis. These times are denoted as low-quality times. Times which are not low-quality are denoted as quality-assured. HFOs occurring during low-quality times are not counted, and the amount of quality-assured time per channel is used as the denominator in computing the HFO rates.

Furthermore, a common averaging technique is applied, for example, per electrode type (depth or surface). The EEG signals across the electrodes are referenced to the common average of all like electrodes, after which an HFO detection is applied to each channel as well as to the common average. One advantage of this method is that it provides better accuracy in distinguishing isolated HFOs from the background by reducing the noise. It also allows identification of periods of diffuse non-neural artifacts, and periods of low-quality collection times.

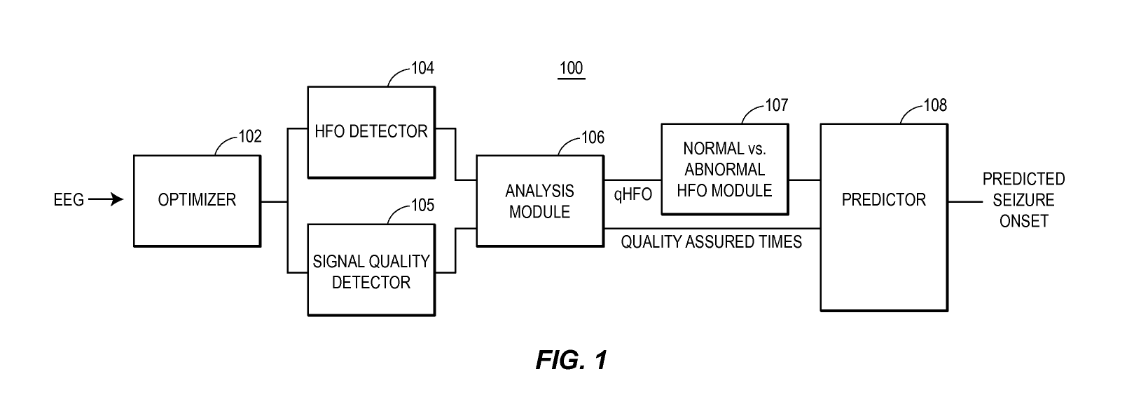

FIG. 1 illustrates an example process 100 for analyzing EEG signals to identify and classify high frequency oscillations indicating seizure onset. EEG data is received from intracranial electrodes, usually 50-120 electrodes. The EEG signal data from each of the electrodes may be continuously collected, over a sampling window of time, which may be minutes, hours, days, or weeks. The typical procedure is for the patient to be admitted to the hospital for 3-14 days with the electrodes implanted, in order to record the EEG continuously during that time and capture spontaneous seizures. The goal is to identify the seizure onset zone (SOZ) and determine whether it can be resected. These patients normally have intractable epilepsy, meaning they have previously failed several antiepileptic medications, and resection is one of the best remaining options for seizure control. Alternatives to resection (i.e. if the SOZ cannot be found or would be unsafe to remove) are further medication trials or neurostimulation, such as vagus nerve stimulation, anterior thalamic stimulation, and closed-loop responsive stimulation. The process herein seeks to improve upon current medical practice by providing additional information to the clinician to help identify and characterize the seizure network. For this process, the EEG data is provided to an optimizer module 102 that processes the received EEG data. Data are collected at sampling rates that allow resolution of the signals of interest, e.g. at least over 1000 Hz for a signal of 500 Hz peak frequency. High frequency signals of interest contain components above 80 Hz, and particularly, components between about 100-1000 Hz, 100-500 Hz, 200-300 Hz, and 100-200 Hz. The optimizer may include various means of referencing, such as common averaging of like electrodes, to improve signal quality, reduce noise, and distinguish high frequency signals that arise from neural activity. This process also generates composite signals that can be used in later steps to assess background activity.

The optimized EEG signal data and composite signals are provided to an HFO detection module 104, which detects HFOs from the received data. In some examples, the HFO detection is performed on the composite signal to produce data indicating when the background activity is likely to generate false positive detections, since HFOs should only occur in a small fraction of electrodes at any one time. In later modules, HFO detection times on the single channels can be compared with the composite detections. The HFO detection module may apply many known HFO detectors, such as the Staba detector, line length algorithms, etc.

The optimized EEG signal data and composite signals are also provided to a signal quality detection module 105, which identifies signals that are unlikely to be neural in origin. This module may include, for example, artifact detectors that identify fast EEG transients that are likely due to technical or electrical effects. It may also include assessment of the signal to noise ratio, identification of periods of flat or volatile EEG signals, or of signals that are too widespread to be HFOs.

The HFO and signal quality detections are provided to an analysis module 106 that compares the times of each detection in order to determine when signals are likely to be neural in origin. This process, for example, may utilize decision trees to identify low-quality epochs of data in which detections should be ignored, and in converse quality-assured epochs in which detections are reliable. The resulting data are quality-assured HFOs (qHFO) and quality-assured epochs in which data are reliable.

The module 107 is an epileptic HFO detector that analyzes each qHFO, as well as the original optimized signal from 102, to determine whether the HFO is likely to be due to epileptic tissue versus a normal HFO that is not associated with epilepsy. This process, for example, may analyze the background signal to identify the colocalization of other EEG activity such as epileptic spikes and amplitude, as well as specific features of the qHFO such as spectral content, amplitude, frequency distribution, line length, etc. The output of this module are ‘epileptic qHFO’ and ‘normal qHFO’.

The module 108 is a predictor of epileptic seizures. It compares the epileptic qHFO from 107 with the quality assured times in 106 in order to determine the rate, time, location and other signal features of HFOs and predict its association with seizures. It may produce, for example, a prediction of the seizure onset zone based upon the number of electrodes that have an anomalously high number of HFOs. It may also predict the likelihood of an oncoming seizure based upon changes in the features of HFOs.

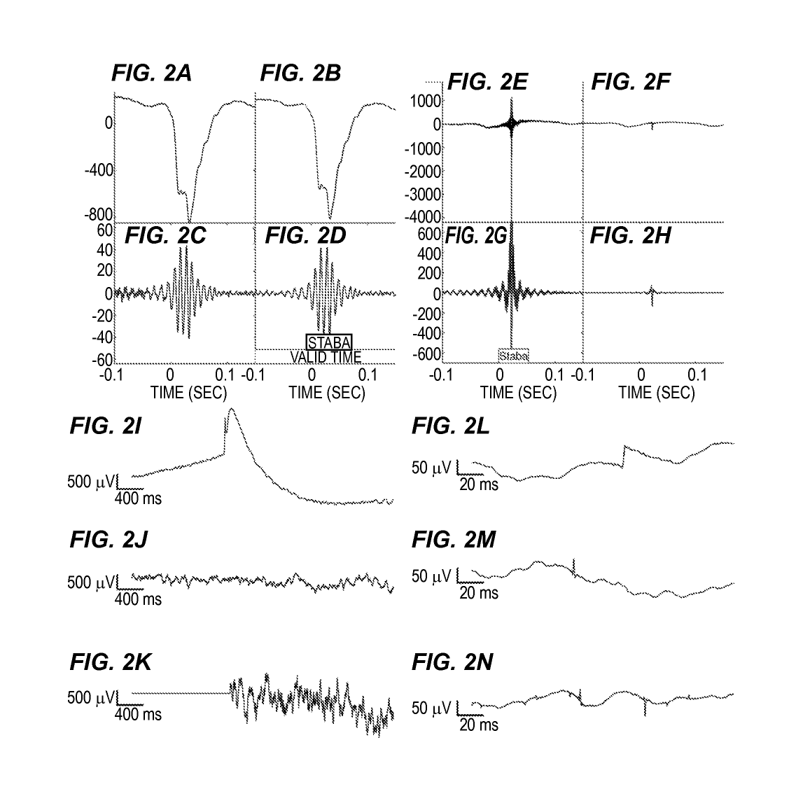

FIG. 2 shows typical examples of detected artifacts and the utility of using optimized signals. Conventional EEG signals lead to both false positive and false negative detections that can be corrected using optimized EEG. Artifact detection and labeling quality-assured times allows removal of HFOs that would have been incorrectly detected. It is noted that in the illustrated implementation of FIG. 1, the entire process does not require any training data per patient, but rather uses a single set of parameters for all patients. In this way, the present techniques can therefore be implemented in a patient agnostic manner.

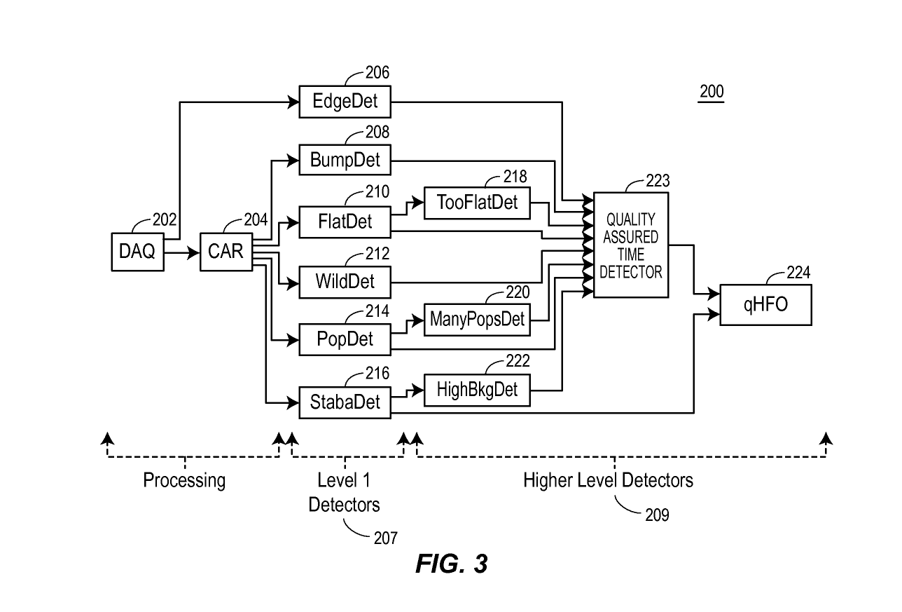

FIG. 3 illustrates a data flow diagram 200 of an example implementation of modules 102, 104, 105, and 106, generating qHFO. A data acquisition (DAQ) device 202 receives the EEG signals from the intracranial electrodes, and the EEG signals are provided to a common average reference (CAR) module 204 that determines an average EEG signal from the collected input signals. The EEG signals from the DAQ are also provided to an edge detector 206, EdgeDet, which independently determines the beginning and end of data epochs where filtering transients should be ignored. The edge detector module 206 is part of a level 1 detector module 207 that performs initial signal analysis prior to a high level detector module 209.

The CAR module 204 computes the common average references in order to optimize each recorded channel and generate the composite signal for further testing. Both the composite signal and each CAR-optimized channel are passed to an array of HFO and signal quality detectors 207. The BumpDet detector module 208 detects slow transients such as FIG. 2I. The FlatDet detector 210 detects periods in which the background activity becomes much lower in amplitude, such as FIG. 2J,K. The WildDet detector 212 identifies volatile (wild) fast activity such as the second half of FIG. 2K. The PopDet detector 214 identifies EEG ‘pops’ (e.g., fast transient shifts or Fast DC-shifts) such as in FIG. 2L, M, N. The StabaDet detector 216 is the implementation of the Staba detector. Each of these detectors is performed on all channels of CAR-optimized data as well as on the composite signal. In addition, there are three additional detectors based upon the results of the FlatDet 210, PopDet 214, and StabaDet 216. The TooFlatDet detector 218 and ManyPopsDet detector 220 both identify when there are too many such detections within a certain time window, and identifies the entire time window as low quality. The HighBkgDet detector 222 analyzes the results of the StabaDet on the composite signal, and identifies any detected HFO on that signal as a poor quality time for all channels included in that composite.

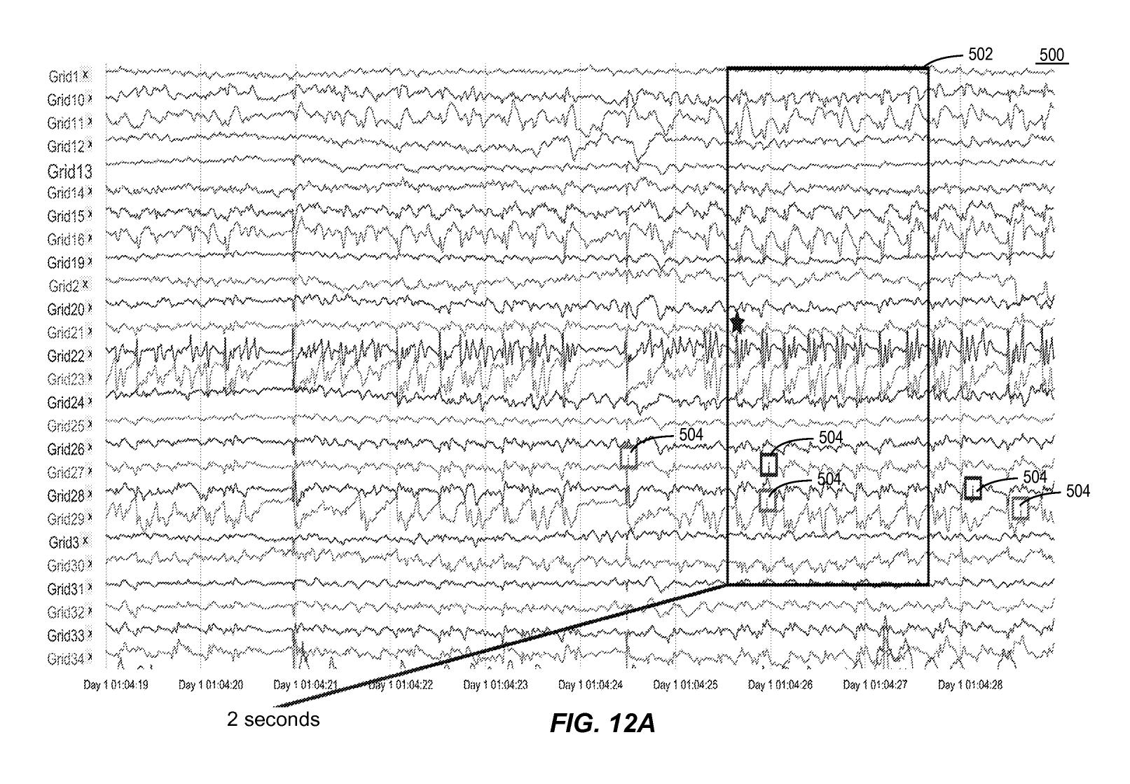

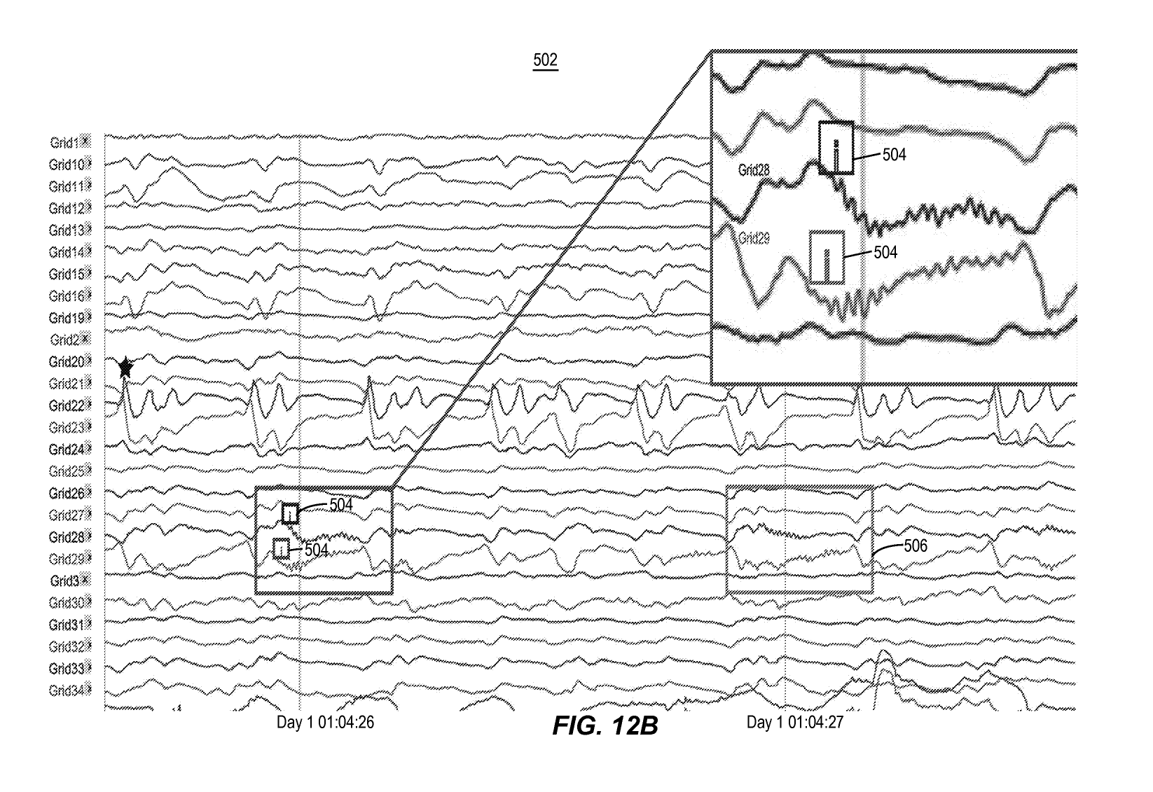

All outputs are then provided to the Quality Assured Time Detector 223, which compares the times of the HFOs from the StabaDet with the signal quality detections. All HFOs that do not occur during any of the detectors of poor signal quality are labeled qHFO, the enhanced HFO data. The outputs of the qHFO are timestamps for each electrode channel that indicate the onset and offset of the original HFO. These data 224 are in the format that allow them to be displayed on all standard EEG viewing software, thus providing a method for integration into the clinical workflow. An example enhanced display is shown in FIGS. 12A and 12B.

The implementation of FIG. 3 is an example. Additional or fewer modules may be used depending on the configuration. For example, another implementation uses simply the DAQ and only some of the level-1 module detectors (e.g., the PopDet module that identifies “pops” i.e., fast transients in the signal and BkgStabaDet module that runs a Staba algorithm on the common average reference signal and determines if an artifact exists) feeding the quality assured time detector that, along with the StabDet level module detector, feeds the qHFO module. Even this reduced module implementation, using only two modules, is able to achieve considerable gains in signal analysis.

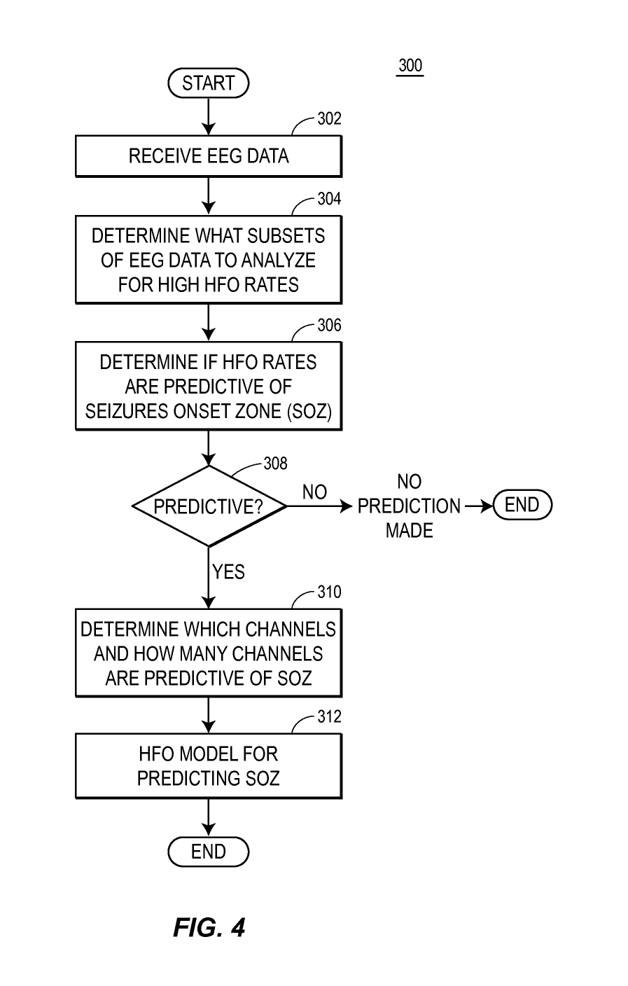

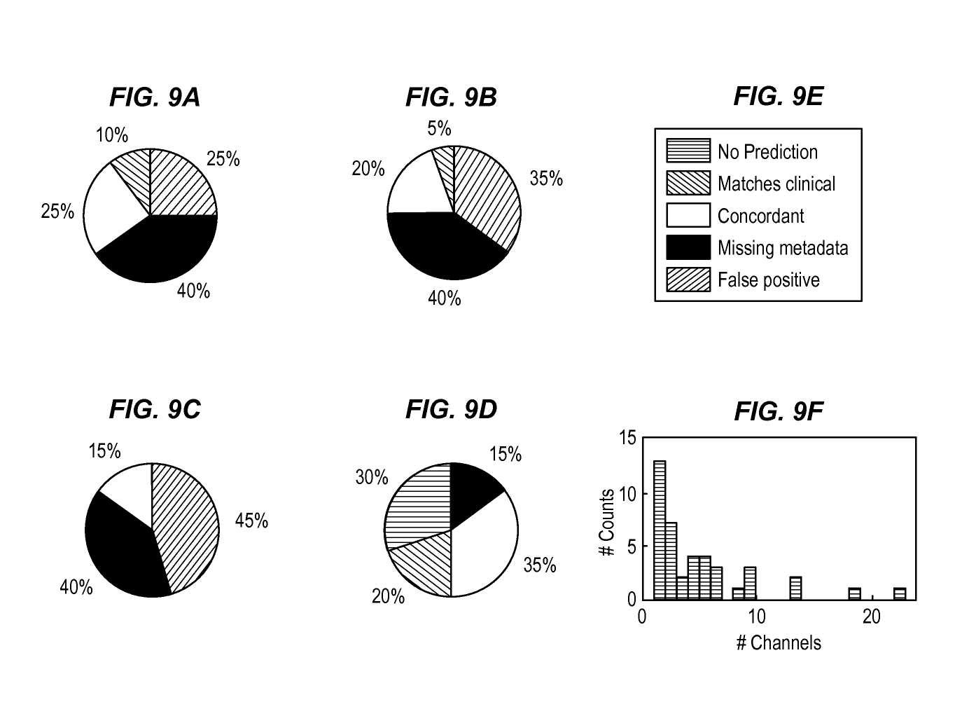

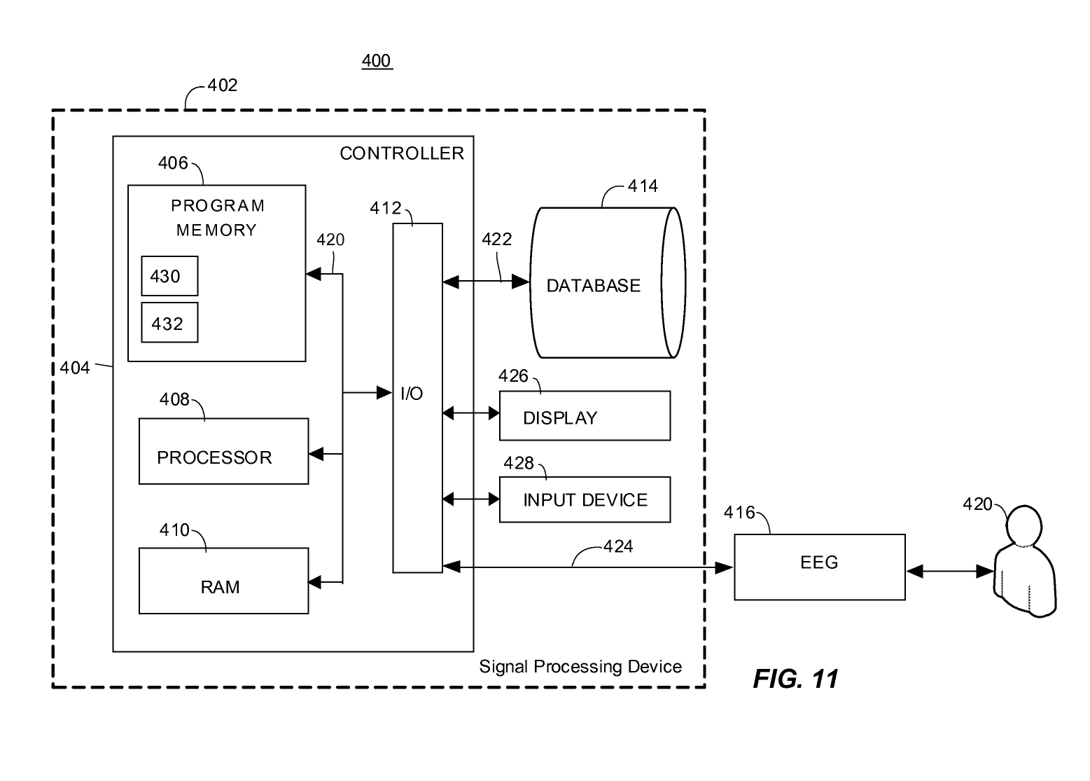

Another aspect of the present teachings is a technique to determine when the HFO rates are predictive of seizure and the extent of the seizure onset zone (SOZ). An example predictive modeling process 300 (108 in FIG. 1), as implemented by the system of FIG. 11, is shown in FIG. 4. The example process (302) receives the qHFO and quality time data from FIG. 3 and determines (304) what subsets of data to analyze for determining anomalously high HFO rates. The qHFO rates and signal quality can vary significantly over the course of an inter-ictal segment, and one must assure that the average amount of quality-assured time is high enough to allow comparison across channels. The next step (306, 308) is to determine whether the HFO rates are predictive, and if so, which and how many channels are predicted to be in the SOZ (310). Merely picking the n-channels with highest HFO rates is not guaranteed to be either sensitive or specific (as demonstrated in FIG. 9). To date, there have been no prospective algorithms published to determine which or how many channels to select as being associated with seizure onset zone. The process 300 may include (310) an adaptive procedure using Kernel Density Estimation (KDEs) that requires no training data and is both flexible and precise enough to automatically adjust to the variations between patients while avoiding spurious detections. The result is the generation of a predictive model of the threshold of the HFO rates that identify SOZ (312).

The techniques herein were performed in a sample experiment according to the following. Patients who underwent intracranial EEG monitoring were selected from the IEEG Portal (www.ieeg.org) and from an IRB-approved protocol at the University of Michigan. From the IEEG Portal, all patient data available in May 2014 was searched for the following inclusion criteria: sampling rate of at least 2700 Hz, a recording time of at least two hours, and data recorded with traditional intracranial electrodes (i.e. not microelectrodes). This yielded 21 patients, five of which were then excluded because there were not at least two hours of continuous high quality inter-ictal data. The remaining 16 patients were all recorded at the Mayo Clinic using a Neurolynx amplifier, although not all data were down-sampled to the same frequency. Additionally, data from four consecutive patients at the University of Michigan were recorded at 30,000 Hz using a dedicated amplifier (Blackrock, Salt Lake City) and down-sampled to 3,000 Hz, resulting in total patient population of 20. All patients were adults with refractory epilepsy undergoing long-term monitoring in preparation for resective surgery. Among the full cohort, it is known that 15 of the 20 underwent resective surgery, and surgery information was not available regarding five patients. Among the 15 patients undergoing surgery, 11 had good surgery outcomes, one outcome (died of SUDEP within one year post-surgery), and three had unknown outcomes.

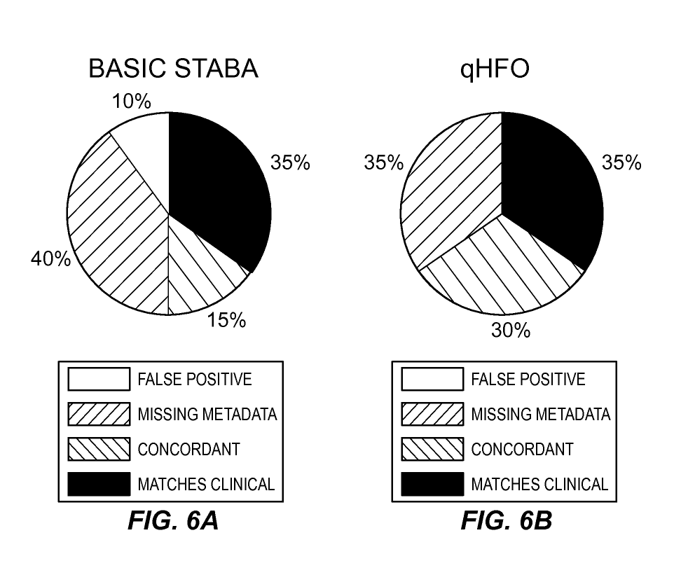

An example of the efficacy of the qHFO method in FIG. 3 is shown in FIG. 6. To quantify how well the HFOs correlated with the SOZ, we compared the channel with the highest HFO rate with the clinical markings of the SOZ, the extent of surgical resection, and long term patient outcomes. It is important to note that this does not use process 107 or 108; it merely evaluates the single channel with highest qHFO rate. When the channel with the most qHFOs was within the marked SOZ, the patient was labeled as “matches clinical”. In the remaining patients, there were some that had insufficient clinical metadata, marked as “missing metadata.” If max channel was resected and the patient had a good outcome, or if the patient had a poor outcome, the patient was marked as “concordant.” If the max channel was outside the resected area in a patient with good outcome, the patient was marked as a “false positive.” There were false positives and a lower number of concordant patients with using the basic Staba detector, both of which improved by implementing the qHFO procedure herein.

Applying the procedure of FIGS. 3 and 4 to predict the SOZ, the subsets of EEG data were determined by identifying quality-assured, inter-ictal (QAII) segments. The EEG data was quantized into 10-minute epochs. QAII segments were defined as a continuous block of epochs, with three features: 1) at least a 3-epoch (30 minute) buffer before and after the epoch including the start of identified clinical seizures, 2) the average (over channels) amount of quality-assured time in each epoch is at least 95%, 3) having a length of at least 11-epochs (110 minutes), based on the minimum amount of time needed for the windowing procedure, described below. Only data within QAII segments were analyzed in this example.

As only four of the 20 patients had complete markings of all clinical seizures, a highly sensitive automated seizure detector was developed using line-length and total power. The results of the automated seizure detector were reviewed to determine whether each detection was a clinical seizure. This procedure exactly replicated the clinical seizures for the four patients with complete markings, and was used to identify the clinical seizures in the other patients. Cumulatively, over 70 QAII segments were determined among the 20 patients, ranging from 110 minutes to nearly 44 hours, with a mean length of 8.6 hours.

To account for variations in the qHFO rate over time, a windowing procedure was employed. The qHFO rates are analyzed over 9-epoch (90 minute) periods, starting at each possible epoch, i.e. every 10 minutes from the start of each QAII segment. Preliminary predictions of the SOZ were made on each 90-minute period using the KDE method described below. Three consecutive 90-minute periods, i.e., one epoch separating the starting time of each adjacent period, were used to determine a semi-final result, with final results being made using all available data per patient. Only channels which were predicted to be in the SOZ in two out of three consecutive preliminary predictions were labeled as SOZ in a semi-final prediction, whereas any channel predicted in any semi-final prediction was included in the final prediction.

In this example, the automated procedure employed an anomaly detection algorithm (identifying whether any channels had HFO rates that were significantly different from the majority of other channels) using KDEs. By comparing all channels, the algorithm first determined whether any channels were anomalous from the background. If not, no reliable prediction can be made and the algorithm aborts. The goal is to maximize specificity rather than sensitivity, as the clinical goal is to minimize the amount of brain tissue resected.

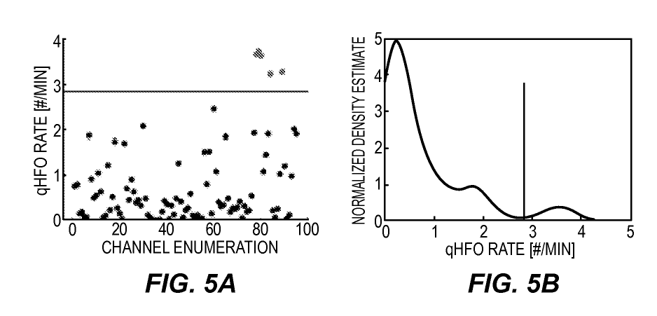

The KDE provides a continuous, non-parametric estimate of the probability distribution from which the given qHFO rates per channel were drawn. A Gaussian-kernel with a bandwidth proportional to the standard deviation of the rates, with a fixed minimum value, was used. In operation, a prediction is made (i.e., at least one channel is anomalously high) only if the density is multi-modal (has multiple peaks) and if the peaks occurring at higher rate are determined to be distinct enough from those at lower rate (based on the minimum density between the peaks, the difference in rates, etc.). Example threshold application is shown in FIGS. 5A and 5B, including the qHFO rates per channel in FIG. 5A and the KDE of the rates in FIG. 5B. The determined threshold is included in both figures. In this example, some fixed thresholds were used uniformly across all 20 patients, such as requiring the median HFO rate to be below a certain value or the determined threshold to be above a certain minimum value. These thresholds were chosen to ensure there were no false positives.

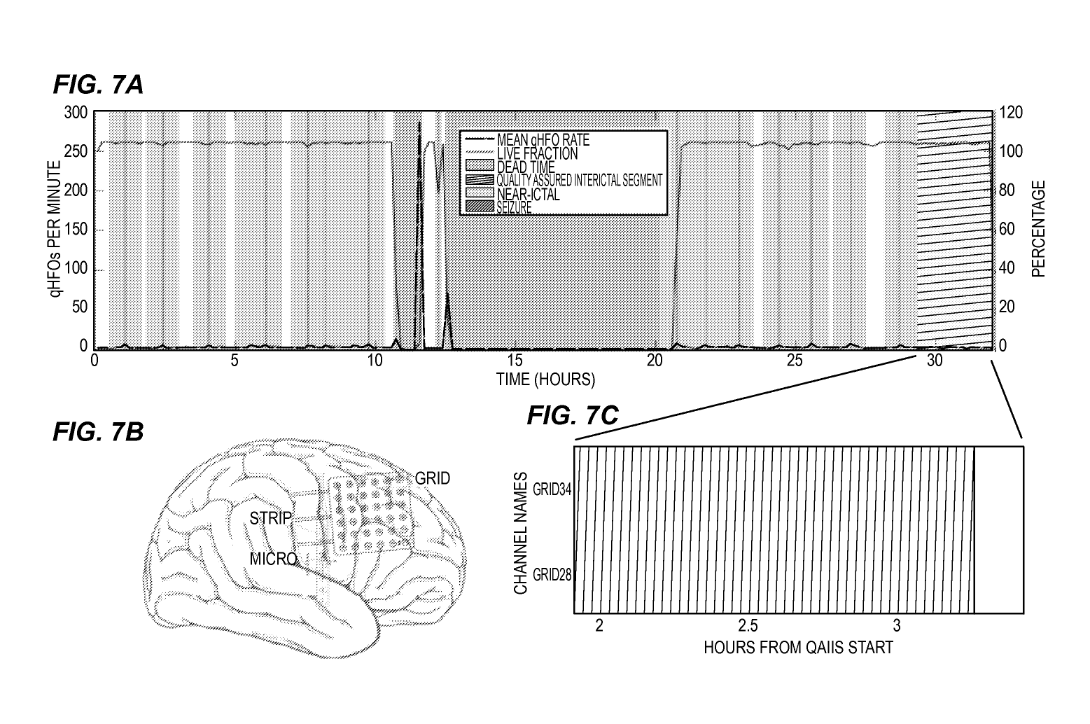

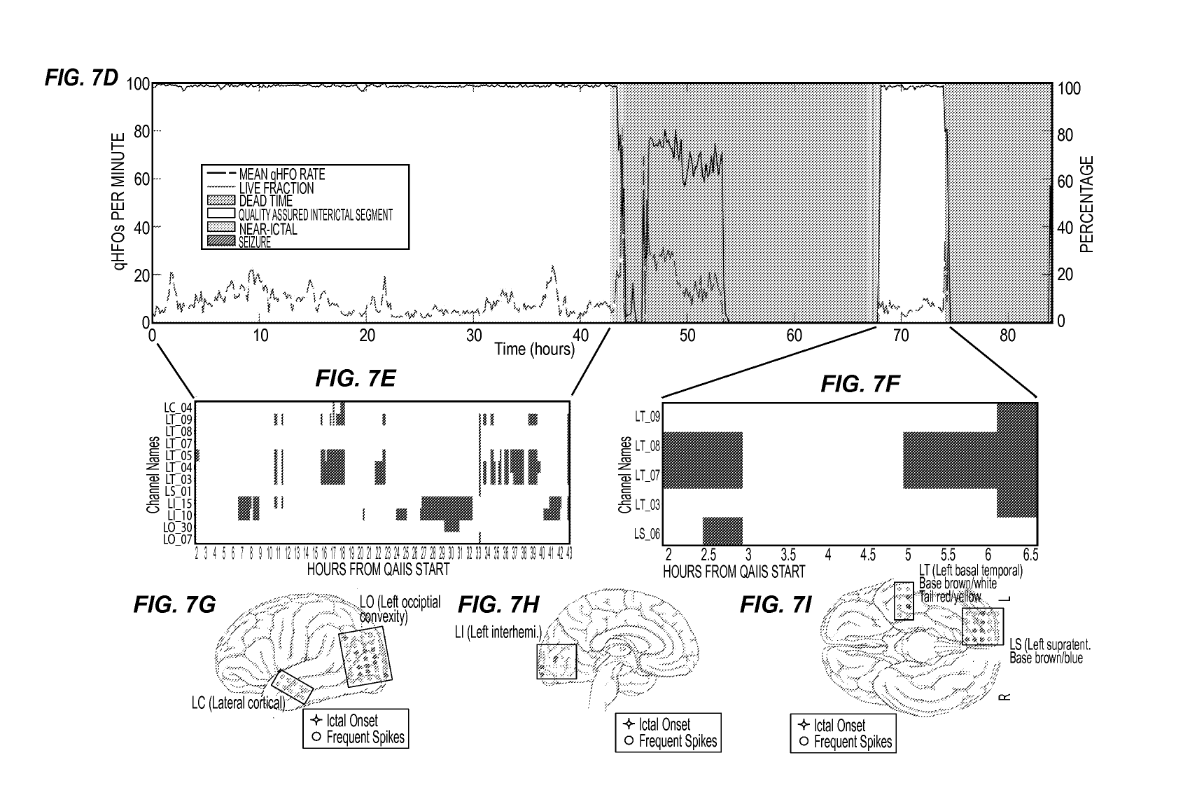

The relationship between the different time scales (full recording session, QAII segments, 90-minute periods and 10-minute epochs) and the location of times resulting in full predictions are shown in FIGS. 7A-7I for two example patients. Note, for both patients the maximum (over channels) qHFO rate per epoch has a few brief extremely high rates during times when the average quality-assured time is below 50%, while at other low-quality times the rate goes to zero. Thus, when too many channels have low-quality data, the qHFO detector is less able to remove all of the artifacts. The restriction to only QAII segments, instead of the full inter-ictal segment, avoided the analysis of the data with these artifacts. In some other cases, low-quality data can cause HFOs not to be identified. Using the length of quality-assured data, instead of the total time length of the data, corrected for this effect. The correction was, however, small due to the high threshold (95%) in defining the QAII segments. Note, the restriction to inter-ictal (i.e. between seizures) segments in this example experiment was chosen to demonstrate that SOZ can be predicted even without recorded seizures. In some models, ictal (i.e. during seizures) data may also be analyzed.

Patient MC 01 (FIG. 7A) does not have a QAII segment until nearly 30 hours after the recording session started, due both to a high number of seizures and due to the recording quality being poor for roughly the middle third of the session. However, during the QAII segment, every 90-minute period predicted exactly the same two channels as anomalously high, which are a subset of the four channels identified clinically as the SOZ. FIGS. 7A-7C correspond to patient MC-01. FIGS. 7D-7I correspond to patient UM-02. This patient has two QAII segments in FIG. 7D. Again the position of the QAII segments is determined by both seizures and data quality. Note, the predicted channels vary significantly over the course of the QAII segments, with apparently two epileptic networks being identified. This result was in concordance with the clinical interpretation, which identified two independent epileptic foci.

For this example, the effect of using the KDE method versus choosing the n-channels with highest qHFO rate is shown in FIGS. 9A-9F. When all predicted channels are within the marked SOZ, the patient was labeled as “matches clinical”, in the remaining patients, there were some that had insufficient clinical metadata, marked as “missing metadata.” If all channels correspond to regions of the brain that were resected and the patient had a good outcome, or if the patient had a poor outcome, the patient was marked as “concordant.” If any channels were outside the resected area in a patient with good outcome, the patient was marked as a “false positive.”

Based on the available meta-data, the KDE method is clearly superior to any forced choice of max−n. The 25% false positive rate for the n=1 case highlights the importance of determining when to predict in addition to just which channels to predict. Note, even though the KDE method often predicts just one channel, it wisely avoids predicting one channel for all cases in which n=1 results in false positives. The n=3 case is the worst possible result: all patients that could be assessed were either false positives (45%) or were labeled as concordant based upon either poor surgery outcomes or a resection of the entire volume covered by the electrodes (15%, each of these patients must be labeled as concordant based upon the definition). Thus, such a method is very unlikely to be useful as a generic algorithm, even though networks of this size are common within the patient population.

The difference in the “missing meta-data” category between the max−n cases and the KDE can be explained as follows: “missing meta-data” is only a possible label if there is a prediction and if the prediction does not exactly match the clinical SOZ. Overall, the KDE method is much more effective than the max−n method, both in its ability to determine when to make a prediction and its ability to vary the number of channels it predictions.

FIG. 9D represents the main performance measure of the complete prospective algorithm in this example. Only six of the patients had data that resulted in no predictions, representing (30±12)% of the population (uncertainty based upon Poisson statistics). Four of the patients had predictions that were subsets of the clinical SOZ, representing (20±7)% of the population. Among the 20 patients, there was not a single prediction that resulted in an obvious false positive.

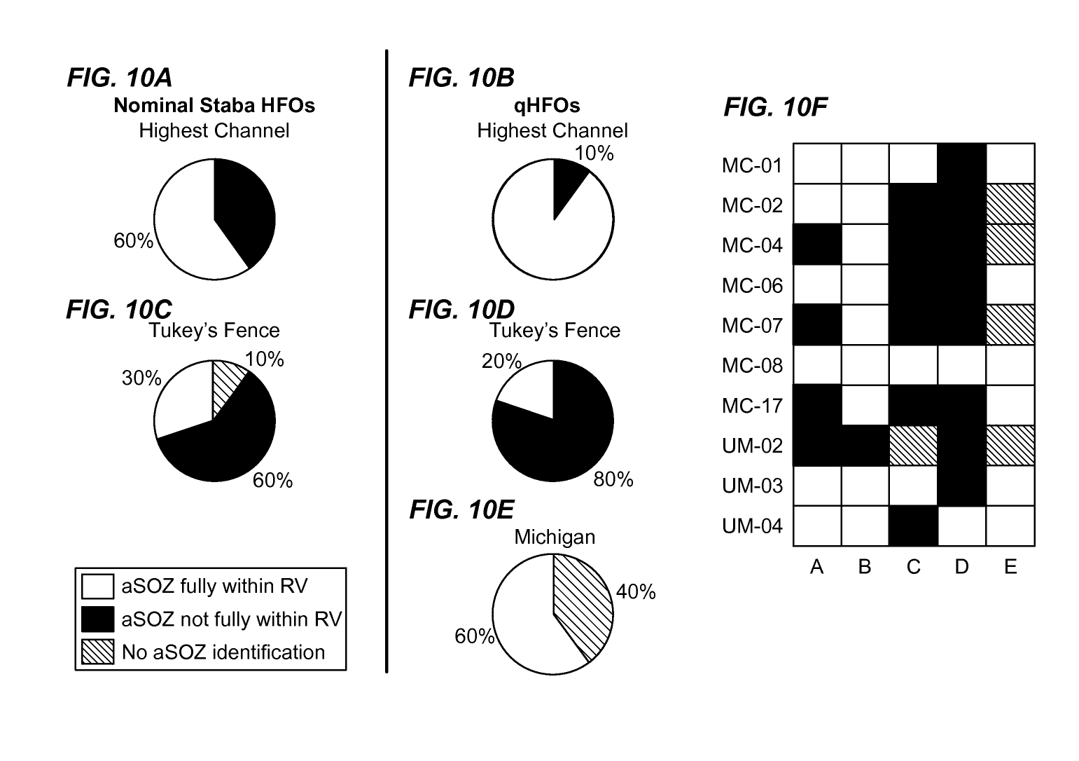

FIGS. 10A and 10C illustrate results for two different nominal Staba HFO techniques, one using highest channel and the other using Tukey's fence ,respectively, to perform signal filtering. Data is depicted showing where the techniques indicate (i) SOZ fully expressed within a resected volume, (ii) seizure onset not fully realized in the resected volume, and (iii) where no seizure onset was identified. FIGS. 10B, 10D, and 10E, illustrate other example techniques, applied to the same data, where FIG. 10E presents the results for the present techniques. For this example, the gold standard to identify the Seizure Onset Zone is when a patient has surgical resection and becomes seizure-free, known as a Class I outcome. This analysis is similar to the examples hereinabove, except all outcome data are available and the results were compared between the algorithmically-determined SOZ and the resected volume (RV) of tissue. We compared results using all HFOs detected with the Staba algorithm (FIGS. 10A and 10C), with the qHFOs (FIGS. 10B, 10D, and 10E). We also compared using the single channel with highest HFO rate (FIGS. 10 A and 10B) with a published method for determining highest HFO rate (namely Tukey's fence, FIG. 10C and FIG. 10D) and with the KDE example of the present techniques (FIG. 10E). These results show that 1) the qHFOs are more specific to epileptic tissue than nominal Staba detections and 2) the KDE example is specific to epileptic tissue while avoiding artifacts. These results were validated with leave-one-out cross-validation, to minimize bias from over-training. The results show, as do the other results herein, an ability of the present techniques to detect HFOs and predict seizure onset zone with no false positives with a single set of parameters for epilepsy. Those results are unprecedented. Plus, the results shown here were obtained with only frequencies of the detected HFOS, i.e., without further data classifying features of HFOs that are known to be specific to epilepsy. Adding that additional information, the present techniques can provide multivariate analyses, with rigorous feature used for more specific HFO algorithms.

The present techniques thus provide the first, fully automated, prospective algorithm to determine a seizure onset zone. The lack of observed false positives indicate that HFOs are a highly specific biomarker of the epileptic network. The specificity is obtained using a process that includes: 1) targeted elimination of artifacts, 2) determining when the HFO rates are predictive of a seizure network, and 3) identification of which and how many channels are predicted to be in the seizure network.

A common average reference (CAR) is used to improve the results, in some examples. Although, more generally, HFOs can be detected using a CAR or not, just as artifacts can be detected using a CAR or not.

Moreover, the determination of when the HFO rates were predictive using the KDE performed better than choosing a fixed number of electrodes, as demonstrated by comparison between the max−n and KDE methods. The max−n procedure resulted in false positives whereas the KDE procedure did not. As a biomarker of epilepsy, it is thus important to be able to identify when the rate of HFOs is sufficiently higher than the rate within normal tissue.

Even with the flexible number of channels being predicted, determination of the exact extent of the seizure network can be challenging. The flexibility of the KDE technique to identify SOZ allowed varying numbers of channels to be examined and a highly-specific algorithm to result. The techniques were able to identify and predict SOZ from inter-ictal (i.e. nonseizure) periods, lasting as little as 110 minutes. This is in stark contrast to typical clinical epilepsy care of these patients, which requires multiple days to identify seizures, which is potentially dangerous to the patient.

This technique also provides novel clinical information about the seizure network that has never been available previously. For example, in one patient it was determined there were two independent seizure networks, involving areas in the subtemporal and lateral parietal lobe. For another patient, networks on both the right and left hemisphere were identified. The correlation between such results and the clinical data will be valuable data regarding seizure networks that have never been previously available to clinicians. Providing these data to clinicians will be a critical step in translating HFO data to clinical care.

In any event, while this experiment focused on a highly-specific identification of individual channels as being within the seizure onset zone, the results suggest that HFOs may also be used as biomarkers of epileptic functional networks, more broadly.

The techniques herein can be incorporated into existing, clinical EEG software. The results support that HFO rates, when used in this manner, are a highly specific biomarker.

FIG. 11 illustrates an example block diagram 400 illustrating the various components used in implementing an example embodiment of the present techniques. A signal processing device 402 (or “signal processor” or “diagnostic device”) is configured to collect EEG data taken from a patient 420 via an EEG device 416 (electrodes not shown) in accordance with executing the functions of the disclosed embodiments. The signal processing device 402 may have a controller 404 operatively connected to a database 414 via a link 422 connected to an input/output (I/O) circuit 412. It should be noted that, while not shown, additional databases may be linked to the controller 404 in a known manner. The controller 404 includes a program memory 406, one or more processors 408 (may be called microcontrollers or a microprocessors), a random-access memory (RAM) 410, and the input/output (I/O) circuit 412, all of which are interconnected via an address/data bus 420. It should be appreciated that although only one processor 408 is shown, the controller 404 may include multiple microprocessors 408. Similarly, the memory of the controller 404 may include multiple RAMs 410 and multiple program memories 406. Although the I/O circuit 412 is shown as a single block, it should be appreciated that the I/O circuit 412 may include a number of different types of I/O circuits. The RAM(s) 410 and the program memories 406 may be implemented as semiconductor memories, magnetically readable memories, and/or optically readable memories, for example. A link 424, which may include one or more wired and/or wireless (Bluetooth, WLAN, etc.) connections, may operatively connect the controller 404 to the EEG device 416 through the I/O circuit 412. In other examples, the EEG device 416 may be part of the signal processing device 402.

The program memory 406 and/or the RAM 410 may store various applications (i.e., machine readable instructions) for execution by the processor 408. For example, an operating system 430 may generally control the operation of the signal processing device 402 and provide a user interface to the signal processing device 402 to implement data processing operations. The program memory 406 and/or the RAM 410 may also store a variety of subroutines 432 for accessing specific functions of the signal processing device 402. By way of example, and without limitation, the subroutines 432 may include, among other things: a subroutine for EEG data from the device 416, a subroutine for optimizing that EEG data such as determining which subsets of EEG data to analyze, a subroutine for determining a common average reference as the composite signal, a subroutine for detecting high-quality HFOs, a subroutine for determining if the high-quality HFO rates are predictive of SOZ, a subroutine for determining which channels and how many channels are predictive of SOZ, and a subroutine of developing an HFO model for predicting SOZ.

The subroutines 432 may also include other subroutines, for example, implementing software keyboard functionality, interfacing with other hardware in the signal processing device 402, etc. The program memory 406 and/or the RAM 410 may further store data related to the configuration and/or operation of the signal processing device 402, and/or related to the operation of the one or more subroutines 432. For example, the data may be data gathered by the device 416, data determined and/or calculated by the processor 408, etc. In addition to the controller 404, the signal processing device 402 may include other hardware resources. The signal processing device 402 may also include various types of input/output hardware such as a visual display 426 and input device(s) 428 (e.g., keypad, keyboard, etc.). In an embodiment, the display 426 is touch-sensitive, and may cooperate with a software keyboard routine as one of the software routines 432 to accept user input. It may be advantageous for the signal processing device 402 to communicate with a medical treatment device, medical data records storage device, or network (not shown) through any of a number of known networking devices and techniques (e.g., through a commuter network such as a hospital or clinic intranet, the Internet, etc.). For example, the signal processing device may be connected to a medical records database, hospital management processing system, healthcare professional terminals (e.g., doctor stations, nurse stations), patient monitoring systems, automated drug delivery systems such as smart pumps, smart infusion systems, automated drug delivery systems, etc. Accordingly, the disclosed embodiments may be used as part of an automated closed loop system or as part of a decision assist system.

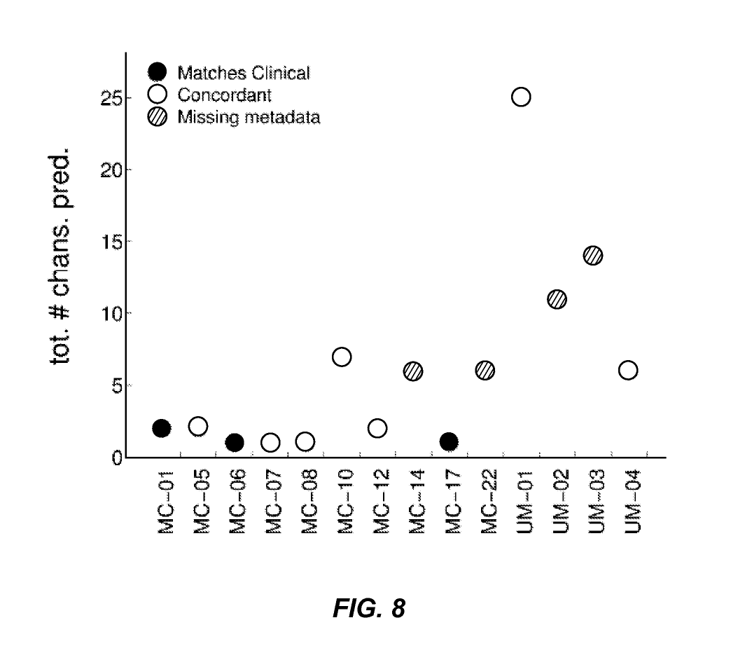

Although depicted as separate entities or components in FIG. 11, it is understood that any or all of the signal processing functionality and/or components of the signal processing device 402 may be combined with an EEG device. In this manner, a system 400 may both gather EEG about the patient 420, e.g., through intracranial electrodes, and process the gathered data to identify and analyze one or more features thereof. Also, although depicted as a single component in FIG. 8, the EEG 416 may include multiple of the same type or different types of brain activity measuring devices.

The present techniques may be integrated into existing EEG devices, including EEG software display devices. The qHFO information, abnormal HFO information, normal HFO information, and predicted seizure onset (whether seizure onset time zone or seizure onset electrode mapping), can be overlayed on existing EEG data and in real time, since these metrics are measured in real time. The particular mode of display is not limited, and these metrics may be displayed alongside EEG data or separate from EEG data. In any event, the present techniques can be integrated into a graphical user interface for display of any of the determinations herein (e.g., qHFO information, abnormal HFO information, normal HFO information, and predicted seizure onset) for review by healthcare professionals.

As discussed, the present techniques include, among other things, the ability to display enhanced electroencephalogram signal data. For example, the present techniques can display EEG signal data enhanced with identified HFO information, including, for example, determinations of quality-assured high frequency oscillations in the electroencephalogram signal data, insufficient quality of the signal the electroencephalogram data, abnormal high frequency oscillations in the electroencephalogram signal data, normal high frequency oscillations in the electroencephalogram signal data, and/or seizure onset. In this way, EEG displays may be enhanced with detected high frequency oscillations, identifying and displaying the time, location, rate and/or features of the high frequency oscillations within a clinical viewing platform. That can provide a physician or caregiver visualized additional information that may be used in the clinical decision making process.

FIG. 12A depicts an electroencephalogram (EEG) 500, that has been modified by the overlay of identified HFO data. The EEG 500 displays the typical data collected from a “grid” of electrodes surgically placed to localize seizure focus. Many of the electrodes show epileptic spikes in one region 502, which shows frequent HFO spikes. An HFO detection technique, as described herein, identified five (5) HFOs 504 in the entire 10 second window of EEG 500 and highlighted those HFOs to create the enhanced EEG 500. The system identified thousands of HFOs over the 2 day study. FIG. 12B illustrates an expanded view of the region 502 showing two of the HFOs 504, which cannot be seen in a normal EEG view even when labeled because the resolution is too low. Across the entire EEG 500, electrodes corresponding to Grid 28 and Grid 29 had the most frequent HFOs 504, and were later found to be the seizure focus.

In the illustrated example, the EEG region 502 also includes a label 506 that identifies a region of HFO-like activity, identify by the techniques herein, but where that activity was below a threshold for detection. Other types of specific information, as discussed in the application, would be depicted in a similar manner in the same enhanced EEG, e.g., using color coding of the different identified data.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method comprising:
continuously receiving, at a signal processing device, neuronal electrical activity signal data taken from a plurality of electrodes and over a sampling window of time;
forming, in the signal processing device, an optimized signal from the neuronal electrical activity signal data;
identifying, in the signal processing device, windows of low-quality signal data collection within the optimized signal and removing the identified windows to form a quality-assured epochs of data collection; and
from the optimized signal, detecting high frequency oscillations in the optimized signal and determining a rate and/or features of high frequency oscillations over the sampling window of time, wherein the rate and/or features of high frequency oscillations are predictive of the onset of a neurological dysfunction in a subject; and
from the optimized signal with detected high frequency oscillations, identifying and displaying the time, location, rate and/or features of the high frequency oscillations within a clinical viewing platform such that a physician or caregiver can visualize this additional information and incorporate it into clinical decision making.

2. The method of claim 1, further comprising:
forming, in the signal processing device, a composite signal from the neuronal electrical activity signal data, wherein the composite signal represents an aggregation of noise and nonneurological electrical activity signal data; and
comparing the optimized signal to the composite signal to determine if high frequency oscillations in the optimized signal are due to neurological brain activity or nonneurological electrical activity or noise.

3. The method of claim 2, further comprising:
determining, from among the high frequency oscillations in the optimized signal that are determined to be due to neurological activity, those high frequency oscillations that are due to neurological dysfunction and those due to normal brain activity.

4. The method of claim 3, further comprising:
determining, from among the high frequency oscillations and other features in the optimized signal that are determined to due to neurological dysfunction, a seizure onset.

5. The method of claim 4, wherein the seizure onset is the region of the brain wherein the seizure initiates, i.e. the seizure onset zone.

6. The method of claim 4, wherein the seizure onset is a location of seizure onset defined by a subset of the plurality electrodes.

7. The system of claim 4, wherein the seizure onset is the temporal onset of a seizure.

8. The method of claim 4, wherein the neurological dysfunction is the occurrence of an epileptic seizure , and wherein the method further comprises:
providing a therapeutic intervention to the identified subset of electrodes in an attempt to preemptively treat the future occurrence of the seizure onset zone.

9. The method of claim 1, wherein the neuronal electrical activity signal data is electroencephalogram signal data.

10. The method of claim 1, wherein the high frequency oscillation signal data is in the range of 80 Hz-1000 Hz.

11. A system comprising:
a processor and a memory, the memory storing instructions that when executed by the processor, cause the processor to:
continuously receive neuronal electrical activity signal data taken from a plurality of electrodes and over a sampling window of time;
form an optimized signal from the neuronal electrical activity signal data;
identify windows of low-quality signal data collection within the optimized signal and removing the identified windows to form a quality-assured epochs of data collection;
from the optimized signal, detect high frequency oscillations in the optimized signal and determining a rate and/or features of high frequency oscillations over the sampling window of time, wherein the rate and/or features of high frequency oscillations are predictive of the onset of a neurological dysfunction in a subject; and
from the optimized signal with detected high frequency oscillations, identify and display the time, location, rate and/or features of the high frequency oscillations within a clinical viewing platform such that a physician or caregiver can visualize this additional information and incorporate it into a clinical decision making.

12. The system of claim 11, the memory storing further instructions that when executed by the processor, cause the processor to:
form a composite signal from the neuronal electrical activity signal data, wherein the composite signal represents an aggregation of noise and nonneurological electrical activity signal data; and
compare the optimized signal to the composite signal to determine if high frequency oscillations in the optimized signal are due to neurological brain activity or nonneurological electrical activity or noise.

13. The system of claim 12, the memory storing further instructions that when executed by the processor, cause the processor to:
determine, from among the high frequency oscillations in the optimized signal that are determined to be due to neurological activity, those high frequency oscillations that are due to neurological dysfunction and those due to normal brain activity.

14. The system of claim 13, the memory storing further instructions that when executed by the processor, cause the processor to:
determine, from the among the high frequency oscillations and other features in the optimized signal that are determined to due to neurological dysfunction, a seizure onset.

15. The system of claim 14, wherein the seizure onset is the region of the brain wherein the seizure initiates, i.e. the seizure onset zone.

16. The system of claim 14, wherein the seizure onset is a location of seizure onset defined by a subset of the plurality electrodes.

17. The system of claim 14, wherein the seizure onset is the temporal onset of a seizure.

18. The system of claim 14, wherein the neurological dysfunction is the occurrence of an epileptic seizure, the memory storing further instructions that when executed by the processor, cause the processor to:
provide a therapeutic intervention to the identified subset of electrodes in an attempt to preemptively treat the future occurrence of the seizure onset zone.

19. The system of claim 11, wherein the neuronal electrical activity signal data is electroencephalogram signal data.

20. The system of claim 11, wherein the high frequency oscillation signal data is in the range of 80 Hz-1000 Hz.

21. A method of displaying electroencephalogram signal data, the method comprising:
receiving the electroencephalogram signal data;
determining at least one of (i) quality-assured high frequency oscillations in the electroencephalogram signal data, (ii) insufficient quality of the signal, (iii) abnormal high frequency oscillations in the electroencephalogram signal data, abnormal high frequency oscillations being due to neurological dysfunction in a subject's brain activity, (iv) normal high frequency oscillations in the electroencephalogram signal data, normal high frequency oscillations being due to normal brain activity in the subject, and (v) seizure onset; and
displaying the electroencephalogram signal data with the determination of (i), (ii), (iii), (iv), and/or (v).

22. The method of claim 21, wherein the displaying the electroencephalogram signal data with determination comprising displaying in real time.

23. A system comprising:
a processor and a memory, the memory storing instructions that when executed by the processor, cause the processor to:
receive the electroencephalogram signal data;
determine at least one of (i) quality-assured high frequency oscillations in the electroencephalogram signal data, (ii) insufficient quality of the signal, (iii) abnormal high frequency oscillations in the electroencephalogram signal data, abnormal high frequency oscillations being due to neurological dysfunction in a subject's brain activity, (iv) normal high frequency oscillations in the electroencephalogram signal data, normal high frequency oscillations being due to normal brain activity in the subject, and (v) seizure onset; and
display the electroencephalogram signal data with the determination of (i), (ii), (iii), (iv), and/or (v).

24. The system of claim 23, the memory storing instructions that when executed by the processor, cause the processor to:
display the electroencephalogram signal data with determination in real time.
